(19) United States
(12) Patent Application Publication
(10) Pub. No.: US 2026/0294953 A1
(43) Pub. Date: Oct. 1, 2026

(54) METHOD OF TREATMENT

(71) Applicant: NUCHIDO LIMITED, Tyne and Wear (GB)

(72) Inventors: Nichola Jane Conlon, Tyne and Wear (GB); Malcolm Philip Young, Tyne and Wear (GB)

(73) Assignee: NUCHIDO LIMITED, Tyne and Wear (GB)

(21) Appl. No.: 19/702,215

(22) Filed: Jun. 9, 2026

Related U.S. Application Data
(63) Continuation of application No. 16/980,207, filed on Mar. 13, 2019.

(30) Foreign Application Priority Data
Mar. 13, 2018 (GB) 1804021.2
Dec. 12, 2018 (GB) 1820236.6

(51) Int. Cl. A61K 31/7084; A61K 31/352; A61K 45/06

(52) U.S. Cl. CPC A61K 31/7084; A61K 31/352; A61K 45/06

(57) ABSTRACT

There is described a method of method of augmentation of plasma which comprises reducing the activity of NAD catabolic and excretory pathway enzymes and/or promoting NAD anabolic pathways, by incorporating into the plasma exogenous NAD or a NAD promoter; or a combination thereof.

### FIELD OF THE INVENTION

The present invention relates to a novel use of plasma or NAD infusion, alongside modification of NAD metabolic networks; and novel methods of treatment related thereto.

More particularly, the present invention relates to the novel use of exogenous NAD or infused blood plasma, given alongside interventions that modify the abundance or activity of enzymes involved in generating NAD from its precursors, degrading NAD or excreting NAD metabolites; the invention also provides a method of treatment related thereto.

### BACKGROUND TO THE INVENTION

Parabiosis is generally a surgical technique that unites the vasculature of two living animals, often to mimic natural instances of shared blood supply, such as conjoined twins or animals that share a placenta in the womb. In heterochronic parabiosis the conjoined animals are typically of different ages,

During heterochronic parabiosis, biomarkers indicative of rejuvenation of the old animal are observed (together with anomalous ageing of the younger one). These results imply that some circulating factor or factors are responsible for the phenotypic changes.

Loffredo et al (Cell 2013; 153 (4): 828-839) proposed that Growth Differentiation Factor 11 is a circulating factor that reverses age-related cardiac hypertrophy. Loffredo et al. demonstrated that after 4 weeks of exposure to the circulation of young mice, cardiac hypertrophy in old mice dramatically regressed, accompanied by reduced cardiomyocyte size and molecular remodelling. Reversal of age-related hypertrophy was not attributable to hemodynamic or behavioural effects of parabiosis, implicating a blood-borne factor. Loffredo identified GDF11 as a circulating factor in young mice that declines with age and found that treatment of old mice to restore GDF11 to youthful levels recapitulated the effects of parabiosis and reversed age-related hypertrophy.

However, this hypothesis has proved controversial given subsequent research, and a consensus presently is that whatever it is that produces the marked phenotypic changes it is not GDF11.

Nonetheless, heterochronal parabiosis does yield phenotypic effects. Similarly, it has been shown that injecting old mice with the plasma portion of blood from young mice improves the memory and ability to learn in the older mice (Villeda et al. Nature Medicine 20:659-663 (2014)) This has led to several commercial initiatives in which plasma from young donors is infused into older patients.

More recently, a number of trials have commenced to study the antiageing benefits of plasma from young, relatively healthy people; and the infusion of plasma from young healthy volunteers into people with Alzheimer's disease.

NAD is a ubiquitous coenzyme, used in energy metabolism, and, we now know, a host of signalling functions. NAD titre is related to the investment a cell makes in DNA and other repair; to calorific restriction; and to the recruitment of senescent cells in the body. NAD concentrations drop exponentially during ageing: from about 8.5 ng/mg protein at birth, to around Ing/mg protein at age 60.

Recent results in vivo show that increasing NAD concentrations in aged mice produces substantial rejuvenation. NAD concentrations are therefore causally involved in ageing phenotypes and recovery from them. Gomes et al (Cell 155, p1624-1638, 2013) showed that, during ageing, there is a specific loss of mitochondrial encoded (oxidative phosphorylation) OXPHOS subunits, which Gomes attributed to an alternate PGC-1α/β-independent pathway of nuclear-mitochondrial communication that is induced by a decline in nuclear NAD+. Deleting SIRT1 (NAD-dependent deacetylase) accelerates this process, whereas raising NAD+ levels in old mice restores mitochondrial function to that of a young mouse in a SIRT1-dependent manner. Gomes et al, showed that 1 week of treatment with a compound that boosts NAD+ levels is sufficient to restore mitochondrial homeostasis and key biochemical markers of muscle health in a 22-month-old mouse to levels similar to a 6-month-old mouse.

Increased NAD levels in young blood plasma over those present in old blood may be sufficient to account for the phenotypic changes apparent in heterochronic parabiosis and young plasma infusion: NAD itself may be the circulating factor responsible for the changes in age-related biomarkers. Increased NAD titres in the recipient are therefore a highly desirable goal if the aim is to improve age-related biomarkers, whether this is achieved by infusion of young plasma or exogenous NAD or the infusion of ingestion of NAD precursors.

Whichever of these routes to causing NAD titre increase is undertaken, supplementation by young plasma, NAD or NAD precursors is unlikely to deliver substantial long-term benefits for several reasons. First, whilst increasing the input to a biological system may result in enhanced output in the short-term, in the longer-term is likely to result in a readjustment of the system to a new equilibrium, resulting in a decline in efficacy (in this case a decline in the NAD level increase as infusion or other supplementation proceeds). Second, there is evidence of saturation of key enzymes that generate NAD from its precursors in such a way that increasing supplementation of either NAD or its precursors above a certain dose suppresses these endogenous enzymes for NAD anabolism. Third, there is evidence for greatly increased excretory pathway activity where NAD or precursor titres are anomalously high, leading to greatly increased excretion of NAD excretory metabolites and the risk of liver damage. These factors suggest that any and all of these routes to increasing NAD in a recipient and so beneficially changing age-related biomarkers do not give lasting enhancement of NAD titres, and could be greatly improved by being given alongside another intervention that modifies NAD anabolic, catabolic and excretory pathway enzymes.

To address these issues, we have invented a “cocktail” of multiple interventions on the enzymes involved in generating destroying and excreting NAD with the aim of greatly potentiating the efficacy and longevity of benefit of infusion of young plasma and NAD. The aim was to potentiate the body's ability to gain benefit from the increased input to the system, by increasing and maintaining the activity of the biological machinery that synthesises NAD, while diminishing the activity of networks that destroy NAD, and those that excrete NAD metabolites. The cocktail (NCD202) was designed to be suitable for oral ingestion and to be given alongside infusion of either young plasma or NAD.

### SUMMARY TO THE INVENTION

It has been surprisingly found that promoting NAD anabolic pathways and reducing the activity of NAD catabolic and excretory pathway enzymes can greatly augment the effects of young plasma infusions and of NAD infusions.

Thus, according to a first aspect of the invention there is provided a method of augmentation of plasma which comprises reducing the activity of NAD catabolic and excretory pathway enzymes and/or promoting NAD anabolic pathways, by incorporating into the plasma exogenous NAD or a NAD promoter; or a combination thereof.

The augmentation of plasma may comprise in vitro plasma augmentation, e.g. prior to the administration of the augmented plasma to an individual, e.g. by infusion; or may comprise in vivo plasma augmentation.

The augmentation of plasma may, in addition, comprise incorporating a NAD precursor into the plasma.

More particularly, the invention provides a method of mitigating the effects of ageing in an individual, said method comprising administering augmented plasma to the individual wherein the plasma is augmented with an effective amount of exogenous NAD or a NAD promoter; or a combination thereof.

The method of mitigating the effects of ageing in an individual may, in addition, comprise incorporating into the plasma a NAD precursor.

The method of the invention will generally comprise administering plasma to an individual by infusion or injection, often the transfer of plasma from a younger individual to an older individual. However, the method of the invention shall not be limited to infusion and, for example, may include plasmapheresis or whole blood pheresis.

The method of the invention may comprise the administration of NAD, or a NAD promoter; and optionally a NAD precursor; and combinations thereof, to one or both of a plasma recipient and a plasma donor.

According to the method of the invention, the augmentation of plasma may comprise the administration of NAD or a NAD promoter; and optionally a NAD precursor; and combinations thereof, to a plasma recipient.

In one aspect of the invention the augmentation of the plasma may comprise the in vivo augmentation, i.e. by administration of NAD, etc. directly to a recipient. In another aspect of the invention the augmentation of the plasma may comprise the in vitro augmentation, i.e. by administration of NAD, etc. directly to the plasma.

Thus, in one aspect the method of the invention comprises the augmentation of plasma by the administration of NAD.

The amount of NAD administered may vary depending upon the mode of administration, etc. Exemplary amounts of NAD which may be in the composition is from about 10 mg to about 1000 mg per day; or from about 50 mg to about 900 mg; or from about 100 mg to about 800 mg; or from about 150 mg to about 700 mg; or from about 200 mg to about 600 mg; or from about 250 mg to about 500 mg.

In another aspect the method of the invention comprises the augmentation of plasma by the administration of a NAD precursor.

NAD precursors are known in the art; and NAD precursors according to the present invention are selected from one or more of niacin (vitamin B₃), tryptophan, quinolinic acid, nicotinic acid mononucleotide (NaMN), nicotinamide riboside (NR), nicotinic acid adenine dinucleotide (NaAD), nicotinamide (NAM), 1-methylnicotinamide (MNA), and nicotinamide mononucleotide (NMN); and derivatives thereof; and any combination thereof. In another aspect of the invention the NAD precursor is a combination comprising two of niacin (vitamin B₃), tryptophan, quinolinic acid, nicotinic acid mononucleotide (NaMN), nicotinamide riboside (NR), nicotinic acid adenine dinucleotide (NaAD), nicotinamide (NAM), 1-methylnicotinamide (MNA), and nicotinamide mononucleotide (NMN); and derivatives thereof.

The amount of NAD precursor administered may vary depending upon the nature of the NAD precursor, the mode of administration, etc. Exemplary amounts of NAD precursor which may be in the composition are from about 10 mg to about 1000 mg per day; or from about 50 mg to about 900 mg; or from about 100 mg to about 800 mg; or from about 150 mg to about 700 mg; or from about 200 mg to about 600 mg; or from about 250 mg to about 500 mg.

In another aspect the method of the invention comprises the augmentation of plasma by the administration of a NAD promoter.

A NAD promoter may comprise one or more of a NAMPT upregulator, a NADase downregulator, a NNMT (nicotinamide N-methyltransferase) downregulator, an upregulator of NMNATs 1-3 (nicotinamide mononucleotide adenylyltransferase), a Cx43 (connexin 43) inhibitor, a CD73 upregulator, a CD157 downregulator, a 5′ AMP-activated protein kinase (AMPK) upregulator, a NR kinase1/2 (NRK1/2) upregulator, a NARPT upregulator, a quinolinate phosphoribosyl transferase (QPRT) upregulator, a NAD synthase 1 (NADSyn1) upregulator, a miRNA-34a downregulator, a purine nucleoside phosphorylase (PNP) upregulator and a NQO1 upregulator; and any combination thereof.

It will generally be understood that the term “downregulator” shall mean an inhibitor or suppressor; and the term “upregulator” shall mean an activator or promoter.

According to one aspect of the invention the NAD promoter is a NAMPT upregulator.

An NAMPT upregulator is an agent that acts to increase the expression of nicotinamide phosphoribosyltransferase. NAMPT upregulators according to the present invention are selected from one or more of phenylephrine, trichostatin A, quercetin (including derivatives of quercetin, such as, 3, 5, 7, 3′, 4′-pentahydroxyflavon, EMIQ isoquercitrin, quercetin 3-O-glucoside, quercetin 3-O-rhamnoside; quercetin 3-O-rhamnozyl-(1→6)-glucoside (rutin); quercetin-3-O-beta-D-glucuronide and 3-methyl quercetin), retinoic acid, pokeweed mitogen, cis-resveratrol, trans-resveratrol, melatonin, troxrutin, b-hydroxy-beta-methyl-butyrate, leucine, apigenin, curcumin, myricetin, genistein, (-)-epigallocatechin-3-gallate (EGCG), kaempferol, luteolin, fisetin, ellagic acid and catechol; and derivatives thereof; and any combination thereof.

In another aspect of the invention the NAMPT upregulator is a combination comprising at least two of phenylephrine, trichostatin A, quercetin (including derivatives of quercetin, such as, 3, 5, 7, 3′, 4′-pentahydroxyflavon, EMIQ isoquercitrin, quercetin 3-O-glucoside, quercetin 3-O-rhamnoside; quercetin 3-O-rhamnozyl-(1→6)-glucoside (rutin); quercetin-3-O-beta-D-glucuronide and 3-methyl quercetin), retinoic acid, pokeweed mitogen, cis-resveratrol, trans-resveratrol, melatonin, troxrutin, b-hydroxy-beta-methyl-butyrate, leucine, apigenin, curcumin, myricetin, genistein, (-)-epigallocatechin-3-gallate, kaempferol, luteolin, fisetin, ellagic acid and catechol; and derivatives thereof.

In this aspect of the invention the NAMPT upregulator is a combination comprising trichostatin A and quercetin.

In another aspect of the invention the NAMPT upregulator is a combination comprising trichostatin A and phenylephrine.

In another aspect of the invention the NAMPT upregulator is a combination comprising trichostatin A and retinoic acid.

In another aspect of the invention the NAMPT upregulator is a combination comprising trichostatin A and pokeweed mitogen.

In another aspect of the invention the NAMPT upregulator is a combination comprising quercetin and phenylephrine.

In another aspect of the invention the NAMPT upregulator is a combination comprising quercetin and retinoic acid.

In another aspect of the invention the NAMPT upregulator is a combination comprising quercetin and pokeweed mitogen.

In another aspect of the invention the NAMPT upregulator is a combination comprising phenylephrine and retinoic acid.

In another aspect of the invention the NAMPT upregulator is a combination comprising phenylephrine and pokeweed mitogen.

In another aspect of the invention the NAMPT upregulator is a combination comprising retinoic acid and pokeweed mitogen.

In another aspect of the invention the NAMPT upregulator is a combination comprising rutin and (-)-epigallocatechin-3-gallate.

In another aspect of the invention the NAMPT upregulator is a combination comprising quercetin and (-)-epigallocatechin-3-gallate.

In another aspect of the invention the NAMPT upregulator is a combination comprising resveratrol and (-)-epigallocatechin-3-gallate.

In another aspect of the invention the NAMPT upregulator is a combination comprising resveratrol, (-)-epigallocatechin-3-gallate and quercetin.

In another aspect of the invention the NAMPT upregulator is a combination comprising resveratrol, (-)-epigallocatechin-3-gallate and rutin.

In another aspect of the invention the NAMPT upregulator is a combination comprising resveratrol, (-)-epigallocatechin-3-gallate, quercetin and rutin.

The amount of NAMPT upregulator administered may vary depending upon the nature of the NAMPT upregulator, the mode of administration, etc. Exemplary amounts of NAMPT upregulator which may be administered are from about 10 mg to about 1000 mg per day; or from about 50 mg to about 900 mg per day; or from about 100 mg to about 800 mg per day; or from about 150 mg to about 700 mg per day; or from about 200 mg to about 600 mg per day; or from about 250 mg to about 500 mg per day.

According to one aspect of the invention the NAD promoter is a NADase downregulator.

A NADase downregulator may be a downregulator of CD38, PARP1 (Poly [ADP-ribose] polymerase 1) and/or SIRTs (NAD-dependent deacetylase sirtuins). CD38 is also known as cyclic ADP ribose hydrolase. It is a glycoprotein found on the surface of many cells. When the method of the invention comprises the administration of a NADase downregulator, the NADase downregulator may be selected from one or more of quercetin, rutin, apigenin, luteolinidin, luteolin, kuromanin, curcumin, myricetin, genistein, (-)-epigallocatechin-3-gallate, kaempferol and luteolin; and derivatives thereof; and any combination thereof.

In another aspect of the invention the NADase downregulator is a combination comprising at least two of quercetin, rutin, apigenin, luteolinidin, luteolin, kuromanin, curcumin, myricetin, genistein, (-)-epigallocatechin-3-gallate, kaempferol and luteolin; and derivatives thereof.

The amount of NADase downregulator administered may vary depending upon the nature of the NADase downregulator, the mode of administration, etc. Exemplary amounts of NADase downregulator which may be in the composition are from about 10 mg to about 1000 mg per day; or from about 50 mg to about 900 mg per day; or from about 100 mg to about 800 mg per day; or from about 150 mg to about 700 mg per day; or from about 200 mg to about 600 mg per day; or from about 250 mg to about 500 mg per day.

According to one aspect of the invention the NAD promoter is a NNMT downregulator.

When the method of the invention comprises the administration of a NNMT downregulator, the NNMT downregulator, may be selected from one or more of tricostatin A, withaferin A, catechin, (-)-epigallocatechin gallate and ellagic acid; and derivatives thereof; and any combination thereof.

In another aspect of the invention the NNMT downregulator is a combination comprising at least two of tricostatin A, withaferin A, catechin, (-)-epigallocatechin gallate and ellagic acid; and derivatives thereof.

The amount of NNMT downregulator administered may vary depending upon the nature of the NNMT downregulator, the mode of administration, etc. Exemplary amounts of NNMT downregulator which may be in the composition are from about 10 mg to about 1000 mg per day; or from about 50 mg to about 900 mg per day; or from about 100 mg to about 800 mg per day; or from about 150 mg to about 700 mg per day; or from about 200 mg to about 600 mg per day; or from about 250 mg to about 500 mg per day.

According to one aspect of the invention the NAD promoter is a NMNATs 1-3 upregulator.

When the method of the invention comprises the administration of a NMNATs 1-3 upregulator, the NMNATs 1-3 upregulator, may be tricostatin A; and derivatives thereof.

The amount of the NMNATs 1-3 upregulator administered may vary depending upon the nature of the NMNATs 1-3 upregulator, the mode of administration, etc. Exemplary amounts of the NMNATs 1-3 upregulator which may be in the composition are from about 10 mg to about 1000 mg per day; or from about 50 mg to about 900 mg per day; or from about 100 mg to about 800 mg per day; or from about 150 mg to about 700 mg per day; or from about 200 mg to about 600 mg per day; or from about 250 mg to about 500 mg per day.

According to one aspect of the invention the NAD promoter is a Cx43 (connexin 43) inhibitor.

When the method of the invention comprises the administration of a Cx43 inhibitor, the Cx43 inhibitor, may be selected from one or more of 18-beta-glycyrrhizic acid, glycyrrhizin, glabridin, ACT1 peptide, resveratrol, 15-delta prostaglandin J2 and puromycin; and derivatives thereof; and any combination thereof.

In another aspect of the invention the Cx43 inhibitor is a combination comprising at least two of 18-beta-glycyrrhizic acid, glycyrrhizin, glabridin, ACT1 peptide, resveratrol, 15-delta prostaglandin J2 and puromycin; and derivatives thereof.

The amount of Cx43 inhibitor administered may vary depending upon the nature of the Cx43 inhibitor, the mode of administration, etc. Exemplary amounts of Cx43 inhibitor which may be in the composition is from about 10 mg to about 1000 mg per day; or from about 50 mg to about 900 mg per day; or from about 100 mg to about 800 mg per day; or from about 150 mg to about 700 mg per day; or from about 200 mg to about 600 mg per day; or from about 250 mg to about 500 mg per day.

According to one aspect of the invention the NAD promoter is a CD73 upregulator.

When the method of the invention comprises the administration of a CD73 upregulator, the CD73 upregulator, may be selected from one or more of acacetin, alprostadil, anisomycin, apigenin, chrysin, dinoprost, luteolin, menadione, myricetin, quercetin and trichostatin A; and derivatives thereof; and any combination thereof.

In another aspect of the invention the CD73 upregulator is a combination comprising at least two of acacetin, alprostadil, anisomycin, apigenin, chrysin, dinoprost, luteolin, menadione, myricetin, quercetin and trichostatin A; and derivatives thereof.

The amount of CD73 upregulator administered may vary depending upon the nature of the CD73 upregulator, the mode of administration, etc. Exemplary amounts of CD73 upregulator which may be in the composition are from about 10 mg to about 1000 mg per day; or from about 50 mg to about 900 mg per day; or from about 100 mg to about 800 mg per day; or from about 150 mg to about 700 mg per day; or from about 200 mg to about 600 mg per day; or from about 250 mg to about 500 mg per day.

According to one aspect of the invention the NAD promoter is a CD157 downregulator.

According to one aspect of the invention the NAD promoter is a 5′ AMP-activated protein kinase (AMPK) upregulator.

When the method of the invention comprises the administration of an AMPK upregulator, the AMPK upregulator, may be selected from one or more of resveratrol, dinitrophenol, quercetin, EMIQ isoquercitrin, rutin, berberine, alpha-lipoic acid, curcumin, genistein, ginsenoside RE, (-)-epigallocatechin gallate, salicylate, astragalus, apigenin, myricetin, kaempferol and luteolin; and derivatives thereof; and any combination thereof.

In another aspect of the invention the AMPK upregulator is a combination comprising at least two of resveratrol, dinitrophenol, quercetin, EMIQ isoquercitrin, rutin, berberine, alpha-lipoic acid, curcumin, genistein, ginsenoside RE, (-)-epigallocatechin gallate, salicylate, astragalus, apigenin, myricetin, kaempferol and luteolin; and derivatives thereof.

The amount of the AMPK upregulator administered may vary depending upon the nature of the AMPK upregulator, the mode of administration, etc. Exemplary amounts of the AMPK upregulator which may be in the composition are from about 10 mg to about 1000 mg per day; or from about 50 mg to about 900 mg per day; or from about 100 mg to about 800 mg per day; or from about 150 mg to about 700 mg per day; or from about 200 mg to about 600 mg per day; or from about 250 mg to about 500 mg per day.

According to one aspect of the invention the NAD promoter is a NR kinase1/2 (NRK1/2) upregulator.

When the method of the invention comprises the administration of a NRK1/2 upregulator, the NRK1/2 upregulator may be selected from one or more of retinoic acid, tricostatin A and resveratrol; and derivatives thereof; and any combination thereof.

In another aspect of the invention the NRK1/2 upregulator is a combination comprising at least two of retinoic acid, tricostatin A and resveratrol; and derivatives thereof.

The amount of NRK1/2 upregulator administered may vary depending upon the nature of the NRK1/2 upregulator, the mode of administration, etc. Exemplary amounts of NRK1/2 upregulator which may be in the composition are from about 10 mg to about 1000 mg per day; or from about 50 mg to about 900 mg per day; or from about 100 mg to about 800 mg per day; or from about 150 mg to about 700 mg per day; or from about 200 mg to about 600 mg per day; or from about 250 mg to about 500 mg per day.

According to one aspect of the invention the NAD promoter is a NARPT upregulator.

According to one aspect of the invention the NAD promoter is a quinolinate phosphoribosyl transferase (QPRT) upregulator.

According to one aspect of the invention the NAD promoter is a NAD synthase 1 (NADSyn1) upregulator.

When the method of the invention comprises the administration of a NADSyn1 upregulator, the NADSyn1 upregulator, may be selected from one or more of vitamin D3 and nadide; and derivatives thereof; and any combination thereof.

In another aspect of the invention the NADSyn1 upregulator is a combination of vitamin D3 and nadide; and derivatives thereof.

The amount of NADSyn1 upregulator administered may vary depending upon the nature of the NADSyn1 upregulator, the mode of administration, etc. Exemplary amounts of NADSyn1 upregulator which may be in the composition are from about 10 mg to about 1000 mg per day; or from about 50 mg to about 900 mg per day; or from about 100 mg to about 800 mg per day; or from about 150 mg to about 700 mg per day; or from about 200 mg to about 600 mg per day; or from about 250 mg to about 500 mg per day.

According to one aspect of the invention the NAD promoter is a miRNA-34a downregulator.

When the method of the invention comprises the administration of a miRNA-34a downregulator, the miRNA-34a downregulator, may be lithium; and derivatives thereof.

According to one aspect of the invention the NAD promoter is a purine nucleoside phosphorylase (PNP) upregulator.

When the method of the invention comprises the administration of a PNP upregulator, the PNP upregulator, may be selected from one or more of quercetin, (-)-epigallocatechin gallate, rutin, tricostatin A, resveratrol and coumestrol; and derivatives thereof; and any combination thereof.

In another aspect of the invention the PNP upregulator is a combination comprising at least two of quercetin, (-)-epigallocatechin gallate, rutin, tricostatin A, resveratrol and coumestrol; and derivatives thereof.

The amount of PNP upregulator administered may vary depending upon the nature of the PNP upregulator, the mode of administration, etc. Exemplary amounts of PNP upregulator which may be in the composition are from about 10 mg to about 1000 mg per day; or from about 50 mg to about 900 mg per day; or from about 100 mg to about 800 mg per day; or from about 150 mg to about 700 mg per day; or from about 200 mg to about 600 mg per day; or from about 250 mg to about 500 mg per day.

According to one aspect of the invention the NAD promoter is a NQO1 upregulator.

When the method of the invention comprises the administration of a NQO1 upregulator, the NQO1 upregulator, may be selected from one or more of selected from one or more of melatonin, trichostatin A, curcumin, retinoic acid, kaempferol, wortmannin, (-)-epigallocatechin gallate, beta-lapachone, hydroquinone, genistein, methyl salicylate, resveratrol, alpha-lipolic acid, 18 alpha glycyrrhetinic acid, apigenin, myricetin, rutin, luteolin, ellagic acid, catechol and quercetin (including derivatives of quercetin); and derivatives thereof; and any combination thereof.

In another aspect of the invention the NQO1 upregulator is a combination comprising at least two of melatonin, trichostatin A, curcumin, retinoic acid, kaempferol, wortmannin, (-)-epigallocatechin gallate, beta-lapachone, hydroquinone, genistein, methyl salicylate, resveratrol, alpha-lipolic acid, 18 alpha glycyrrhetinic acid, apigenin, myricetin, rutin, luteolin, ellagic acid, catechol and quercetin (including derivatives of quercetin); and derivatives thereof.

The amount of NQO1 upregulator administered may vary depending upon the nature of the NQO1 upregulator, the mode of administration, etc. Exemplary amounts of NQO1 upregulator which may be in the composition are from about 10 mg to about 1000 mg per day; or from about 50 mg to about 900 mg per day; or from about 100 mg to about 800 mg per day; or from about 150 mg to about 700 mg per day; or from about 200 mg to about 600 mg per day; or from about 250 mg to about 500 mg per day.

According to one aspect of the invention when the composition comprises a NAMPT upregulator comprising a combination of resveratrol, (-)-epigallocatechin-3-gallate and quercetin, the composition may comprise resveratrol (from about 2 to about 6 parts w/w, e.g. about 4 parts); (-)-epigallocatechin-3-gallate (from about 2 to about 4 parts w/w, e.g. about 3 parts); and quercetin (from about 5 to about 15 parts w/w, e.g. about 10 parts).

According to a further aspect of the invention there is provided exogenous NAD, a NAD precursor or a NAD promoter; or a combination thereof, for use in the augmentation of plasma.

In one aspect of the invention there is provided exogenous NAD for use in the augmentation of plasma.

In another aspect of the invention there is provided a NAD precursor for use in the augmentation of plasma.

In another aspect of the invention there is provided a NAD promoter for use in the augmentation of plasma.

In another aspect of the invention there is provided a combination of exogenous NAD and a NAD precursor or a NAD promoter; for use in the augmentation of plasma.

In another aspect of the invention there is provided a combination of a NAD precursor and a NAD promoter; for use in the augmentation of plasma.

According to this aspect of the invention the NAD promoter may comprise one or more of a NAMPT upregulator, a NADase downregulator, a NNMT (nicotinamide N-methyltransferase) downregulator, an upregulator of NMNATs 1-3 (nicotinamide mononucleotide adenylyltransferase), a Cx43 (connexin 43) inhibitor, a CD73 upregulator, a CD157 downregulator, a 5′ AMP-activated protein kinase (AMPK) upregulator, a NR kinase1/2 (NRK1/2) upregulator, a NARPT upregulator, a quinolinate phosphoribosyl transferase (QPRT) upregulator, a NAD synthase 1 (NADSyn1) upregulator, a miRNA-34a downregulator, a purine nucleoside phosphorylase (PNP) upregulator and NQO1 upregulator; and derivatives thereof; and any combination thereof; for use in the augmentation of plasma.

More particularly, the invention provides exogenous NAD, a NAD precursor or a NAD promoter; or a combination thereof, for use in mitigating the effects of ageing in an individual, which comprises administering augmented plasma to the individual wherein the plasma is augmented with an effective amount of exogenous NAD, a NAD precursor or a NAD promoter; or a combination thereof.

According to this aspect of the invention the NAD promoter may comprise one or more of a NAMPT upregulator, a NADase downregulator, a NNMT (nicotinamide N-methyltransferase) downregulator, an upregulator of NMNATs 1-3 (nicotinamide mononucleotide adenylyltransferase), a Cx43 (connexin 43) inhibitor, a CD73 upregulator, a CD157 downregulator, a 5′ AMP-activated protein kinase (AMPK) upregulator, a NR kinase1/2 (NRK1/2) upregulator, a NARPT upregulator, a quinolinate phosphoribosyl transferase (QPRT) upregulator, a NAD synthase 1 (NADSyn1) upregulator, a miRNA-34a downregulator, a purine nucleoside phosphorylase (PNP) upregulator and a NQO1 upregulator; and derivatives thereof; and any combination thereof; for use in the augmentation of plasma.

The use according to this aspect of the invention will generally comprise use by administering augmented plasma to an individual by infusion or injection, generally the transfer of plasma from a younger individual to an older individual. However, the use of this aspect of the invention shall not be limited to infusion and, for example, may include plasmapheresis or whole blood pheresis.

According to a further aspect of the invention there is provided a composition comprising an effective amount of exogenous NAD, a NAD precursor or a NAD promoter; or a combination thereof, for use in the augmentation of plasma.

More particularly, the invention provides a composition comprising an effective amount of exogenous NAD, a NAD precursor or a NAD promoter; or a combination thereof, for use in mitigating the effects of ageing in an individual, which comprises administering augmented plasma to the individual wherein the plasma is augmented with an effective amount of exogenous NAD, a NAD precursor or a NAD promoter; or a combination thereof, for use in the augmentation of plasma.

According to this aspect of the invention the NAD promoter may comprise one or more of a NAMPT upregulator, a NADase downregulator, a NNMT (nicotinamide N-methyltransferase) downregulator, an upregulator of NMNATs 1-3 (nicotinamide mononucleotide adenylyltransferase), a Cx43 (connexin 43) inhibitor, a CD73 upregulator, a CD157 downregulator, a 5′ AMP-activated protein kinase (AMPK) upregulator, a NR kinase1/2 (NRK1/2) upregulator, a NARPT upregulator, a quinolinate phosphoribosyl transferase (QPRT) upregulator, a NAD synthase 1 (NADSyn1) upregulator, a miRNA-34a downregulator, a purine nucleoside phosphorylase (PNP) upregulator and a NQO1 upregulator; and derivatives thereof; and any combination thereof; for use in the augmentation of plasma.

A specific composition which may be mentioned comprises an effective amount of a combination of one or more NAMPT upregulators; one or more AMPK upregulators; and one or more NAD precursors.

According to this aspect of the invention the composition may comprise an effective amount of a combination of resveratrol, quercetin, rutin, apigenin, alpha-lipoic acid and nicotinamide riboside.

A further composition according to this aspect of the invention may comprise an effective amount of a combination of apigenin, rutin, (-)-epigallocatechin-3-gallate, niacinamide and alpha-lipoic acid.

The compositions of the invention may also include one or more bioavailability enhancers or skin penetration enhancers. Such bioavailability enhancers or skin penetration enhancers shall include, but shall not be limited to, DMSO, decyl methyl sulfoxide, N-dodecyl pyrrolidone, decanol, dodecanol, an organic acid such as oleic acid, zinc, vitamin C and piperine (Bioperine®) or the like; and combinations thereof. In one aspect of the invention the bioavailability enhancers or skin penetration enhancers include zinc, vitamin C and piperine (Bioperine®); and combinations thereof.

The composition according to this aspect of the invention will generally include an acceptable excipient.

The effects of ageing may include age related skin conditions, skin conditions related to sun exposure, skin conditions related to pollution exposure, skin conditions related to oxidative stress, and skin conditions related to lifestyle choices, such as diet, alcohol and/or smoking. In addition, the compositions of the invention may be advantageous in the mitigation, alleviation or improvement of skin conditions related to inflammatory skin disorders and skin conditions related autoimmune disease skin disorders. The compositions of the invention may be advantageous in the mitigation, alleviation or improvement of other age-related conditions, such as, but not limited to, increased frailty, loss of resilience, loss of muscle strength, loss of muscle endurance, loss of energy, loss of cognitive sharpness, loss of memory, etc. More specifically, the compositions of the invention may be advantageous in the mitigation, alleviation or improvement of other age-related conditions, such as, but not limited to, atherosclerosis and cardiovascular disease, cancer, arthritis, cataracts, osteoporosis, type 2 diabetes, hypertension and Alzheimer's disease; the incidence of which increases with aging.

In addition, the method and compositions of the invention may be advantageous in the treatment, alleviation or improvement of skin conditions related to inflammatory skin disorders and skin conditions related autoimmune disease skin disorders.

Current augmentation of plasma by NAD enhancement can comprise intermittent NAD IV infusion, administration of an oral NAD precursor, as herein described, or a combination of both NAD IV infusion and NAD precursor.

However, whilst NAD IV infusions will enhance NAD by an external method that does not use the NAD generating processes intrinsic to cells, such infusions may also inhibit the natural cellular production of endogenous NAD, due to negative feedback inhibition of the enzymes responsible for transforming NAD precursors into NAD and/or the excretion of the surplus precursor. This risks lower endogenous NAD production during and after the NAD IV infusion.

Compositions of the present invention may also be used alongside NAD IV infusions to counteract this negative feedback effect by maintaining optimal enzyme ratios/enzyme activity in the cell, thus counteracting the possible negative impact of the infusion.

In the same way, supplementation alone with NAD and/or an NAD precursor may also saturate the enzymes, thus reducing the cells ability to synthesise NAD, resulting in poor in vivo conversion of the endogenous and exogenous NAD precursors.

Compositions of the present invention prevent this enzyme saturation and allow the cell to maximally utilise the NAD precursors for greater and more efficient conversion to NAD.

Compositions of the present invention will also downregulate the NNMT enzyme. NNMT methylates the NAD precursor NAM and this methylated NAM is then excreted from the body. Consequently, the excreted NAM is unavailable to be converted back into NAD via the salvage pathway. Therefore, downregulation of NNMT will maintain higher net in vivo NAD because more NAM is available to enter salvage pathway to be converted back to NAD.

Compositions of the present invention are advantageous in that they rebalance endogenous NAD-related enzymes toward NAD production; and away from NAD catabolismand/or NAD excretion; thus generating higher NAD levels. Even with direct NAD IV infusion or NAD precursor supplementation, failing to prevent NAD catabolismand/or NAD excretion will result in much lower NAD enhancement than could otherwise be achieved.

Therefore, compositions of the present invention may also be used during NAD IV infusion and/or NAD precursor supplementation to ensure that the endogenous production of NAD is not inhibited and to ensure that cells are primed to be able to maximally convert exogenous and endogenous NAD precursor into NAD.

Thus, according to this aspect of the invention there is provided a method of mitigation, alleviation or improvement of the effects of ageing in a host, said method comprising the enhancement of endogenous and exogenous NAD and/or NAD precursors by the administration of an effective amount of a composition as herein described.

The enhancement of endogenous and exogenous NAD and/or NAD precursors as herein described would prevent catabolismand excretion of NAD and/or NAD precursors.

The method according to this aspect of the invention may comprise the administration of an effective amount of a composition of the invention in isolation, i.e. as the sole source of endogenous NAD or NAD precursor, or as an adjunct therapy accompanying NAD IV infusion and/or administration of an oral NAD precursor.

Age related skin conditions that may be mitigated, alleviated or improved, shall include, but shall not be limited to, one or more of sagging, wrinkles, skin elasticity, skin ageing, skin moisture, wounds, acne, skin darkening, skin whitening, pigmentation, age-spots, loss of radiance, puffiness, uneven skin tone, redness, rosacea, loss of barrier function, loss of skin resilience, loss of firmness, stretch-marks, cellulite and dryness.

Skin conditions related to sun exposure that may be treated, alleviated or improved, include, but shall not be limited to, one or more of actinic keratoses, freckles, lentigines or age spots, moles, photosensitivity, polymorphous light eruption, seborrheic keratoses, skin cancer (such as melanoma, squamous cell carcinoma, basal cell carcinoma), solar elastosis or wrinkles and sun burn.

Skin conditions related to inflammatory skin disorders that may be treated, alleviated or improved, include, but shall not limited to, one or more of acne, asteatotic eczema, atopic dermatitis, contact dermatitis, discoid eczema, eczematous drug eruptions, erythema multiforme, erythroderma, gravitational/varicose eczema, hand eczema, keratosis lichenoides chronica, lichen nitidus, lichen planus, lichen simplex, lichen striatus, mycosis fungoides, pityriasis lichenoides, psoriasis, seborrheic dermatitis, Stevens-Johnson Syndrome, toxic epidermal necrolysis and vasculitis.

Skin conditions related autoimmune disease skin disorders that may be treated, alleviated or improved, include, but shall not limited to, one or more of alopecia areata, bullous pemphigoid, dermatomyositis, dystrophic epidermolysis bullosa, eosinophilic fasciitis, pemphigus vulgaris, psoriasis, pyoderma gangrenosum, scleroderma, systemic lupus erythematosus and vitiligo.

Neurological disorders that may be treated, alleviated or improved, include, but shall not limited to, anxiety, panic disorders, depression, schizophrenia, cognition, pain and neurodegenerative disorders, such as, Multiple Sclerosis, dementia, Alzheimer's disease, Parkinson's disease, stroke, traumatic brain injury and the like.

Cardiovascular diseases shall include, but shall not be limited to, diseases caused by vascular contraction, such as cerebrovascular spasmodic disease following subarachroid haemorrhage or cerebral infarction, cardiovascular spasmodic disease, hypertension, kidney diseases, cardiac infarction, angina, arrhythmia, portal hypertension in association with cirrhosis and varicosity in association with cirrhosis. Ischemia and reperfusion injury, congestive cardiac failure, bradycardia and tachycardia. Diseases caused by vascular dilation, such as chronic headache, e.g., hemicrania, tension headache, headache of the mixed type, cluster headaches, haemorrhoid and cardiac diseases.

Autoimmune disorders and related immune disorders shall include, but shall not be limited to, systemic lupus erythematosus (SLE), rheumatoid arthritis, non-glomerular nephrosis, psoriasis, chronic active hepatitis, ulcerative colitis, Crohn's disease, Behcet's disease, chronic glomerulonephritis, chronic thrombocytopenia purpura, and autoimmune haemolytic anaemia.

The composition of the present invention may be administered orally or parenterally; or may comprise controlled, modified or extended release formulations comprising suitable treatment amounts of the desired active components in the form of powders, granules, sterile parenteral solutions or suspensions, oral solutions or suspensions, oil water emulsions as well as implants and microencapsulated delivery systems.

### Parenteral Administration

Thus, according to one aspect of the invention there is provided the composition as herein described for parenteral administration.

When the composition of the invention is administered parenterally, it may be in the form of an intramuscular, intravenous, subcutaneous, intraperitoneal, local or transdermal bolus injection or continuous infusion.

Suitable formulations for topical application, e.g., to the skin and eyes, include aqueous solutions, suspensions, ointments, creams, gels, sprayable formulations, transdermal patch or bandage e.g., for delivery by aerosol or the like. Such topical delivery systems will in particular be appropriate for dermal application, for prophylactic use in sun creams, lotions, sprays and the like. They are thus particularly suited for use in topical, including cosmetic, formulations well-known in the art. Such formulations may contain solubilisers, stabilizers, tonicity enhancing agents, buffers and preservatives.

Transdermal devices may be in the form of a bandage comprising a backing member, a reservoir containing the composition of the invention optionally with carriers, optionally a rate controlling barrier to deliver the composition of the invention to the skin of the host at a controlled and predetermined rate over a prolonged period of time, and means to secure the device to the skin.

### Oral Administration

Thus, according to one aspect of the invention there is provided the composition as herein described for oral administration.

When the composition of the invention is administered orally, it may be in the form of tablets or capsules.

The compositions of the invention can be made up in a solid form including capsules, tablets, pills, granules, powders, food bar or confectionery; or in a liquid form including solutions, suspensions or emulsions or in the form of a syrup, linctus, elixir, a liquid beverage, such as a yoghurt drink, or a foodstuff, such as a yoghurt.

The compositions can be subjected to conventional operations such as sterilization and/or can contain conventional inert diluents, lubricating agents, or buffering agents, as well as adjuvants, such as preservatives, stabilizers, wetting agents, emulsifiers and buffers etc.

Typically, when the compositions are in the form of tablets or capsules, e.g. gelatin capsules, the compositions may comprise the active components, i.e. NAD, a NAD precursor or a NAD promoter; or a combination thereof;
together with
   a) diluents, e.g., lactose, dextrose, sucrose, mannitol, sorbitol, cellulose and/or glycine;
   b) lubricants, e.g., silica, talcum, stearic acid, its magnesium or calcium salt and/or polyethyleneglycol; for tablets also;
   c) binders, e.g., magnesium aluminium silicate, starch paste, gelatin, tragacanth, methylcellulose, sodium carboxymethylcellulose and/or polyvinylpyrrolidone; if desired;
   d) disintegrants, e.g., starches, agar, alginic acid or its sodium salt, or effervescent mixtures; and/or
   e) absorbents, colourants, flavours and sweeteners.

Tablets may be either film coated or enteric coated according to methods known in the art.

Suitable compositions for oral administration include an effective amount of the active components described herein in the form of tablets, lozenges, aqueous or oily suspensions, dispersible powders or granules, food bar, confectionery, solution, emulsion, hard or soft capsules, a syrup, linctus, elixir, a liquid beverage, such as a yoghurt drink, or a foodstuff, such as a yoghurt.

Compositions intended for oral use can be prepared according to any method known in the art for the manufacture of effective compositions; and such compositions can contain one or more additional agents selected from the group consisting of sweetening agents, flavouring agents, colouring agents and preserving agents in order to provide elegant and palatable preparations.

Tablets contain the composition comprising the active components herein described, in admixture with non-toxic orally acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets may be uncoated or coated by known techniques to delay disintegration and absorption in the gastrointestinal tract and thereby provide a sustained action over a longer period. For example, a time delay material such as glyceryl monostearate or glyceryl distearate can be employed.

Formulations for oral use can be presented as hard gelatin capsules wherein the composition comprising the active components is mixed with an inert solid diluent, for example, calcium carbonate, calcium phosphate or kaolin, or as soft gelatin capsules wherein the composition comprising the active components is mixed with water or an oil medium, for example, peanut oil, liquid paraffin or olive oil.

The soft capsule can be prepared using techniques well known in the art. For example, soft capsules are typically produced using a rotary die encapsulation process. Active agent formulations are fed into the encapsulation machine by gravity. In an embodiment, the formulation comprises pharmaceutical excipients such as olive oil, gelatin, glycerin, purified water, beeswax yellow, sunflower lecithin, silicon dioxide, titanium dioxide, F. D. & C Blue 1 and F. D. & C Red 4, microcrystalline cellulose, hypromellose, vegetable magnesium stearate, and/or silica.

A capsule shell can comprise one or more plasticizers such as glycerin, sorbitol, sorbitans, maltitol, glycerol, polyethylene glycol, polyalcohols with 3 to 6 carbon atoms, citric acid, citric acid esters, triethyl citrate and combinations thereof. In an embodiment, the plasticizer is glycerin.

In addition to the plasticizer(s), the capsule shell can include other suitable shell additives such as opacifiers, colourants, humectants, preservatives, flavourings, and buffering salts and acids.

Opacifiers are used to opacify the capsule shell when the encapsulated active agents are light sensitive. Suitable opacifiers include, but not limited to, titanium dioxide, zinc oxide, calcium carbonate and combinations thereof. In an embodiment, the opacifier is titanium dioxide.

Colourants can be used to for marketing and product identification and/or differentiation purposes. Suitable colourants include synthetic and natural dyes and combinations thereof.

Humectants can be used to suppress the water activity of the softgel. Suitable humectants include glycerin and sorbitol, which are often components of the plasticizer composition. Due to the low water activity of dried, properly stored softgels, the greatest risk from microorganisms comes from molds and yeasts. For this reason, preservatives can be incorporated into the capsule shell. Suitable preservatives include alkyl esters of p-hydroxy benzoic acid such as methyl, ethyl, propyl, butyl and heptyl (collectively known as “parabens”) or combinations thereof.

Selection of a particular effective dose can be determined (e.g., via clinical trials) by a person skilled in the art based upon the consideration of several factors which will be known to the person skilled in the art, such as, the disorder to be treated, alleviated or improved; the nature and severity of the disorder being treated, the body mass of the host; and the like. The precise dose employed in the treatment, alleviation improvement of the disorder may also depend upon the route of administration. Effective doses can be extrapolated from dose-response curves derived from in vitro or animal model test systems.

However, in general, satisfactory results may be obtained at a daily dosage of the composition of the invention of from about 0.1 to about 500 mg/kg body weight; or from about 1 to about 400 mg/kg; or from about 1 to about 300 mg/kg; or from about 1 to about 200 mg/kg; or from about 1 to about 100 mg/kg; or from about 10 to about 50 mg/kg; administered, for example, in divided doses up to three or four times a day, e.g. twice daily, or in sustained release form.

It is often practical to administer the daily dose of the composition of the invention at various hours of the day. The amount of the active composition administered may depend on such factors as the solubility of the active composition, the formulation used, subject condition (such as weight), and/or the route of administration.

The present invention will now be described by way of example only.

### EXPERIMENTAL

### Infusion Augmentation Trial Protocol

### Measurement of NAD

NAD was measured in peripheral blood mononuclear cells before and after administration of the intervention to one male subject. In brief, blood was extracted via venepuncture and PBMCs were isolated using density gradient separation. NAD was measured in the PBMC fraction using the NAD+/NADH-Glo assay kit (Promega). NAD measurements were adjusted for protein concentration using a standard BCA assay.

### Establishment of Baseline NAD

Cellular NAD concentrations within the body display diurnal variation. To accurately measure changes in NAD, these natural fluctuations in baseline NAD must be considered. Prior to administration of the intervention, baseline NAD fluctuations were investigated by obtaining blood samples from the subject over a period of 12 hours (8a.m. to 8p.m.) at a frequency of every 2 hours. This was repeated on 3 separate days and NAD was measured as described above. This was again repeated on 3 separate days on which infusion of young plasma or exogenous DNA was undertaken. These data were used to inform a suitable timepoint for NAD measurement during the augmentation intervention. This was decided to be 2 μm, a timepoint where NAD was consistently lower.

### Intervention Ingredients and Dosage

The intervention comprised a proprietary cocktail (NCD202). NCD202 comprised resveratrol, isoquercitrin and EGCG.

| | Required |
|---|---|
| Component | dose |
| Quercetin | 1000 mg |
| Resveratrol | 400 mg |
| EGCG | 300 mg |

### Dosing Regimen

Interventions were administered orally at 8: 30 am (overnight fasted) for 7 consecutive days. A blood sample was obtained each day at 2 μm during the intervention and NAD was measured as described above.

After the intervention, a period of washout was then observed for 7 consecutive days during which no intervention was administered. At the end of the washout period, NAD was measured to check if it had returned to baseline levels. Upon confirmation that baseline levels of NAD were re-established, a further intervention was then administered for a further 7 days and NAD measurements repeated.

Dosing was designed to provide data on NAD baseline titres without any intervention; with infusion of young plasma with no other intervention; infusion of exogenous NAD alone; infusion of young plasma accompanied by administration of NCD202; infusion of exogenous NAD accompanied by administration of NCD202; infusion of young plasma accompanied by administration of placebo; and infusion of exogenous NAD accompanied by administration of placebo.

1. A method of augmentation of plasma which comprises reducing the activity of NAD catabolic and excretory pathway enzymes and/or promoting NAD anabolic pathways, by incorporating into the plasma exogenous NAD or a NAD promoter; or a combination thereof.

2. A method of mitigating the effects of ageing in an individual, said method comprising administering augmented plasma to the individual wherein the plasma has been augmented by a method according to claim 1.

3. A method according to claim 1 wherein the augmentation of plasma comprises incorporating a NAD precursor into the plasma.

4. A method according to claim 1 wherein the method comprises administering plasma to an individual by parabiosis, infusion, plasmapheresis or whole blood pheresis.

5. A method according to claim 1 wherein the augmentation of plasma comprises the administration of NAD, a NAD precursor or a NAD promoter; or a combination thereof, to a plasma recipient, a plasma donor and/or directly to the plasma.

6. A method according to claim 5 wherein the augmentation of plasma comprises the administration of NAD.

7. A method according to claim 6 wherein the amount of NAD administered is from about 10 mg to about 1000 mg per day.

8. A method according to claim 1 wherein the augmentation of plasma comprises the administration of a NAD precursor.

9. A method according to claim 8 wherein the NAD precursor is selected from one or more of niacin (vitamin B₃), tryptophan, quinolinic acid, nicotinic acid mononucleotide (NaMN), nicotinamide riboside (NR), nicotinic acid adenine dinucleotide (NaAD), nicotinamide (NAM), 1-methylnicotinamide (MNA), and nicotinamide mononucleotide (NMN); and derivatives thereof; and any combination thereof.

10. (canceled)

11. A method according to claim 9 wherein the amount of NAD precursor administered is from about 10 mg to about 1000 mg per day.

12. A method according to claim 1 wherein the augmentation of plasma comprises the administration of a NAD promoter.

13. (canceled)

14. (canceled)

15. A method according to claim 1 wherein the NAD promoter is a NAMPT upregulator is selected from one or more of phenylephrine, trichostatin A, quercetin (including derivatives of quercetin, such as, 3, 5, 7, 3′, 4′-pentahydroxyflavon, EMIQ isoquercitrin, quercetin 3-O-glucoside, quercetin 3-O-rhamnoside; quercetin 3-O-rhamnozyl-(1→6)-glucoside (rutin); quercetin-3-O-beta-D-glucuronide and 3-methyl quercetin), retinoic acid, pokeweed mitogen, cis-resveratrol, trans-resveratrol, melatonin, troxrutin, b-hydroxy-beta-methyl-butyrate, leucine, apigenin, curcumin, myricetin, genistein, (-)-epigallocatechin-3-gallate, kaempferol, luteolin, fisetin, ellagic acid and catechol; and derivatives thereof; and any combination thereof.

16. (canceled)

17. A method according to claim 1 wherein the composition comprises an effective amount of a combination of one or more NAMPT upregulators; one or more AMPK upregulators; and one or more NAD precursors.

18. A method according to claim 1 wherein the composition comprises an effective amount of a combination of resveratrol, quercetin, rutin, apigenin, alpha-lipoic acid and nicotinamide riboside.

19. A method according to claim 1 wherein the composition comprises an effective amount of a combination of apigenin, rutin, (-)-epigallocatechin-3-gallate, niacinamide and alpha-lipoic acid.

20. (canceled)

21. A method according to claim 131 wherein the NAD promoter is a NADase downregulator.

22. A method according to claim 21 wherein the NADase downregulator is selected from one or more of quercetin, rutin, apigenin, luteolinidin, luteolin, kuromanin, curcumin, myricetin, genistein, (-)-epigallocatechin-3-gallate, kaempferol and luteolin; and derivatives thereof; and any combination thereof.

23.-137. (canceled)

138. A method of administering to an individual exogenous NAD and an effective amount of a combination of apigenin, rutin, (-)-epigallocatechin-3-gallate, niacinamide and alpha-lipoic acid.

139. A method according to claim 138 wherein the method includes mitigating the effects of ageing in an individual by administering to said individual exogenous NAD and an effective amount of a combination of apigenin, rutin, (-)-epigallocatechin-3-gallate, niacinamide and alpha-lipoic acid to said individual.
